(19) United States
(12) Patent Application Publication
(10) Pub. No.: US 2024/0074988 A1
(43) Pub. Date: Mar. 7, 2024

(54) DEVICES AND METHODS FOR THE TRANSDERMAL DELIVERY OF PSILOCYBIN

(71) Applicant: Remy Biosciences, Inc., Irvine, CA (US)

(72) Inventors: Brent Cahill, Laguna Beach, CA (US); Mark Burleson, Irvine, CA (US); Bruce Cahill, Irvine, CA (US)

(21) Appl. No.: 17/824,901

(22) Filed: May 26, 2022

Related U.S. Application Data
(60) Provisional application No. 63/193,585, filed on May 26, 2021.

(51) Int. Cl. A61K 9/70; A61K 31/00; A61K 31/4045; A61K 31/675

(52) U.S. Cl. CPC A61K 9/7084; A61K 31/4045; A61K 31/658; A61K 31/675

(57) ABSTRACT

Monolithic-style dermal patches and reservoir-style dermal patches are provided. The dermal patches are capable of delivering to a patient, consumer, user, or human subject, one or both of psilocybin and psilocin, preferably with one or more cannabinoids, such as cannabidiol (CBD).

### CROSS-REFERENCE TO RELATED APPLICATIONS

This application claims the full Paris Convention benefit of, and priority to United States provisional patent application serial numbered 63/193,585, which is incorporated by this reference as if fully set forth herein in its entirety.

### BACKGROUND

Dermal patches and related medical devices and methods of the present disclosure can deliver one or more pharmaceutical agents, such as psilocybin, psilocin, and cannabidiol (CBD).

Psilocybin is a small molecule consisting of an indole group, a phosphate group covalently bound to the six-membered benzene ring of the indole group, and an ethylamine group covalently bound to the five-membered pyrrole ring of the indole group. Indole has a bicyclic structure, consisting of a six-membered benzene ring fused to a five-membered pyrrole ring. Psilocybin is a stable prodrug of the active metabolite, psilocin. Psilocybin is an agonist of the human 5-HT2A-receptor (see, Grob, Danforth, Greer (2011) Arch. Gen. Psychiatry. 68:71-78; Fricke, Kargbo, Hoffmeister (2020) Chem. Eur. J. 26:8281-8285). Psilocybin has received Breakthrough Therapy designation by the FDA, for treating depression and for treating major depressive disorder (see, Fricke et al, supra; Krediet, Breeksema, Vermetten (2020) Int. J. Neuropsychopharmacology. 23:385-400).

Delivery of pharmaceutical agents can be accomplished by monolithic-style dermal patches (also known as “drug-in-adhesive” devices), and by reservoir-style dermal patches with liquid carriers and a porous membrane, such as a hydrophobic rate-controlling membrane.

Monolithic-style dermal patches can take the form of a sandwich, where the face that is exposed to the atmosphere has a backing, where the opposite face has a release liner, and where the filling of the sandwich takes the form of a matrix that includes an adhesive and one or more pharmaceutical agents. Prior to applying the patch to the skin, the release liner is removed and discarded. Regarding reservoir-style dermal patches, the reservoir also contains a liquid carrier and a gelling agent. The reservoir can be defined by a backing and by a permeable membrane, which together assume a “ravioli” conformation. The permeable membrane is optionally coated with an adhesive that mediates binding of the adhesive to the user's skin. On one side of the adhesive is the permeable membrane, and on the other side is a release liner. Prior to applying the patch to the skin, the release liner is removed and discarded.

Clinical studies have suggested that psilocybin, in combination with psychological counseling, can provide relief to patients suffering from anxiety, depression, and post-traumatic stress disorder (Hutten, Kuiypers (2019) Frontiers in Psychiatry. 10: Article 672).

The present disclosure provides pharmaceutical compositions or formulations, dermal patches, components of said dermal patches, and methods for manufacturing said dermal patches. The present disclosure also provides methods for using dermal patches, for example, methods for attaching a dermal patch to the skin, and methods for treating the user with pharmaceutical agents that can relieve one or more disorders, such as depression, anxiety, and pain.

### BRIEF DESCRIPTIONS OF THE DRAWINGS

FIG. 1 is a side cross-sectional view of a reservoir-style dermal patch for delivering pharmaceutical agents to a wearer's skin.

FIG. 2 is a side cross-sectional view of a monolithic-style dermal patch for delivering pharmaceutical agents to a wearer's skin.

### SUMMARY OF THE PRESENT DISCLOSURE

The present disclosure provides a reservoir-type dermal patch that includes: (i) A backing and a hydrophilic, porous membrane, (ii) Wherein the backing and hydrophilic, porous membrane are attached to one another to define reservoir, wherein the reservoir comprises a pharmaceutical formulation, (iii) Wherein the hydrophilic, porous membrane comprises a first surface that is on the side of the hydrophilic, porous membrane that contacts the backing, (iv) Wherein the hydrophilic, porous membrane further comprises a second surface that is on the side of the hydrophilic, porous membrane that does not contact the backing and, wherein the second surface is coated with a skin adhesive, (v) Wherein the dermal patch further comprises a release liner that contacts the second surface that is coated with the skin adhesive, (vi) And wherein the pharmaceutical formulation comprises one or both of psilocybin and psilocin.

In another aspect, what is provided is the above reservoir-type dermal patch, wherein the formulation further comprises one or more antioxidants, said one or more antioxidants consisting of an antioxidatively effective concentration of an ascorbyl palmitate, or consisting of an antioxidatively effective concentration of ascorbic acid, or consisting of a combination of both an antioxidatively effective concentration of ascorbyl palmitate plus ascorbic acid, wherein the formulation includes psilocybin, and wherein said one or more antioxidants is capable of reducing the rate of oxidation of psilocybin.

In yet another aspect, what is provided is the above dermal patch, wherein the formulation further comprises one or more antioxidants, wherein said one or more antioxidants comprises an antioxidatively effective concentration of an ascorbyl palmitate, or an antioxidatively effective concentration of ascorbic acid, or a combination of both an antioxidatively effective concentration of ascorbyl palmitate plus ascorbic acid, wherein the formulation includes psilocin, and wherein said one or more antioxidants is capable of reducing the rate of oxidation of psilocin.

Furthermore, what is encompassed by the present disclosure is the above reservoir-type dermal patch, wherein regarding the formulation, the sum of the concentrations of psilocybin and psilocin is one of about 0.02% by weight (unit of wt./wt.), about 0.04% by weight, about 0.06% by weight, about 0.08% by weight, about 1.0% by weight, about 2.0% by weight, about 4% by weight, about 6% by weight, about 8% by weight, about 10% by weight, about 12% by weight, about 14% by weight, or about 16% by weight, wherein the unit of weight is weight of psilocybin in a gram of formulation were the formulation does not contain psilocin, or weight of psilocin in a gram of formulation where the formulation does not contain psilocybin, or weight of the sum of psilocybin plus psilocin where the formulation contains both psilocybin and psilocin.

In yet another aspect, what is contemplated is the above dermal patch, wherein the reservoir contains a hydrogel, as well as the above dermal patch, wherein the reservoir does not contain any hydrogel.

In yet another aspect, what is embraced is the above reservoir-style dermal patch, wherein the reservoir contains a penetration enhancer, and wherein the penetration enhancer is at a concentration of about 2% (wt./wt.), about 4%, about 6%, about 8%, about 10%, about 12%, about 14%, about 16%, about 18%, or about 20%. Further provided is the above dermal patch, wherein the reservoir does not contain any penetration enhancer, or wherein the reservoir contains under one percent penetration enhancer (wt./wt.).

Also embraced is the above dermal patch, wherein the formulation contains one or more penetration enhances selected from transcutol, dimethylsulfoxide (DMSO), azone, oleic acid, dihydromyricetin, isopalmitate, propylene glycol, and isopropyl myristate.

In pharmaceutical agent embodiments, what is provided is the above dermal patch, wherein the formulation comprises psilocin and one or more cannabinoids, or wherein the formulation comprises psilocybin and one or more cannabinoids, or wherein the formulation comprises both psilocin and psilocybin and one or more cannabinoids.

The present disclosure provides a monolithic-type dermal patch that includes: (i) A backing, (ii) A matrix comprising a skin adhesive, an adhesion/viscosity modifier, and a pharmaceutical formulation, wherein the pharmaceutical formulation comprises one or more of psilocybin, psilocin, a derivative of psilocybin, a derivative of psilocin, a cannabinoid, and an antioxidant, (iii) A release liner, wherein the release liner has an inner face and a outer face, and wherein the inner face is substantially in contact with the matrix, wherein the outer face does not contact the matrix, and wherein said release liner can be peelably removed from the surface of said adhesive matrix.

Also embraced is the above monolithic-type dermal patch, wherein the adhesion/viscosity modifier is a mineral oil or a silicone fluid.

In addition, what is embraced is the above monolithic-dermal patch, wherein the mineral oil or the silicone fluid is present in an amount ranging from about one percent to about ten percent of the weight of the matrix.

In mineral oil embodiments, what is provided is the above monolithic-type dermal patch, wherein the mineral oil has a molecular weight ranging from 200-400 Daltons, from 200-500 Daltons, from 300-500 Daltons, from 300-600 Daltons, from 400-600 Daltons, from 400-700 Daltons, from 500-700 Daltons, from 500-800 Daltons, from 600-800 Daltons, from 600-900 Daltons, from 700-900 Daltons, from 700-1000 Daltons, or from any combination of said ranges.

In silicone fluid embodiments, what is provided is the above monolithic-type dermal patch, wherein the silicone fluid comprises hydroxyl group (—OH) end-capped polydimethylsiloxanes having a kinematic viscosity at 20 degrees C. ranging from 100 cSt to about 1000 sCt, or wherein the kinematic viscosity at 20 degrees C. is about 20 cSt, or about 100 cSt, or about 350 cSt, or about 1000 cSt, or about 12,500 cSt.

In polyisobutylene adhesive embodiments, what is contemplated is the above monolithic-type dermal patch, wherein the skin adhesive is a polyisobutylene that is supplied in mineral oil, or wherein the skin adhesive is a polyisobutylene that is not supplied in mineral oil.

In adhesive blend embodiments, what is embraced is the above monolithic-type dermal patch, wherein the skin adhesive comprises a blend of acrylic adhesive and polyisobutylene adhesive.

In amine-compatible silicone adhesive embodiments, what is contemplated is the above monolithic-type dermal patch, wherein the skin adhesive is an amine-compatible silicone adhesive.

In penetration enhancer embodiments, what is embraced is the above monolithic-type dermal patch, that comprises a penetration enhancer, and wherein said penetration enhancer comprises one or more of oleic acid, isopropyl palmitate (IPP), DMSO, 1,2 propylene glycol, and isopropyl myristate (IPM). Also, what is provided is the above monolithic-type dermal patch, wherein the amount of penetration enhancer preferably ranges from zero to about ten percent by weight of the matrix.

As used herein, including the appended claims, the singular forms of words such as “a,” “an,” and “the” include their corresponding plural references unless the context clearly dictates otherwise. All references cited herein are incorporated by reference to the same extent as if each individual patent, and published patent application, as well as figures, drawings, sequence listings, compact discs, and the like, was specifically and individually indicated to be incorporated by reference.

### Scientific Units and Non-Limiting Definitions

The word, “substantially.” The word, “substantially,” for example, as with the release liner of a dermal patch that is “substantially in contact” with the matrix layer of a dermal patch, can mean that at least 60%, at least 70%, at least 80%, at least 90%, or about 100%, of one face of the matrix is in direct contact with the release liner.

The word “about.” The following refers to a list of numbers, where the list has numbers of progressively increasing values. A preferred meaning of the term “about” is any value in a range, where the range is defined by the half-way point between the following two values: (1) The stated middle value and the next lowest value, and (2) The stated middle value and the next highest value. The following illustrates the meaning of the above meaning of “about.” The term “about 12 grams” means all values between 11 grams and 13 grams. This meaning is derived from the list of “about values” that is disclosed in the above paragraph.

To provide another non-limiting definition of “about,” the following concerns the example of, “about 20, about 40, about 60, about 80, about 90, about 100, about 150, about 200, about 250, about 300, about 350, about 400, about 450, about 500, about 550, about 600, about 650, about 700, about 750, about 800, about 850, about 900, about 950, about 1000, about 2000, about 3000, about 4000, about 5000, about 6000, about 7000, about 8000, about 9000, about 10,000, or about 12,500 cSt.” In this context, the term “about” can mean plus or minus 5% of the stated value, or plus or minus 10% of the stated value.

Viscosity units. Millipascals seconds, abbreviated as mPa-s, is a unit of viscosity. 100 Centipoise equals 1 Poise. 1 Centipoise=1 mPa s (Millipascal Second). 1 Poise=0.1 Pa s (Pascal Second). These definitions are from V&P Scientific, San Diego, CA. According to another source, the unit of viscosity is, Pascal−second (Pa−S) (see, Khonsari, Booser (208) Applied Tribology, Bearing Design and Lubrication, 2nd ed.).

Kinematic viscosity. Kinematic viscosity can be expressed in terms of the unit, centistokes (cSt). Kinematic viscosity (ν) is the ratio of the viscous force to the inertial force or fluid density (ρ). This ratio is calculated as follows: ν=μ/ρ. Kinematic viscosity has SI units of (meters squared) divided by seconds. The physical unit for kinematic viscosity is stokes (St) or centistokes (cSt). See, Martyr, Plint (2012) Tribology, Fuel, and Lubrication Testing in Engine Testing, 4th ed.

### DETAILED DESCRIPTIONS OF THE DRAWINGS, WITH PART NUMBERS REFERRING TO THE DRAWINGS

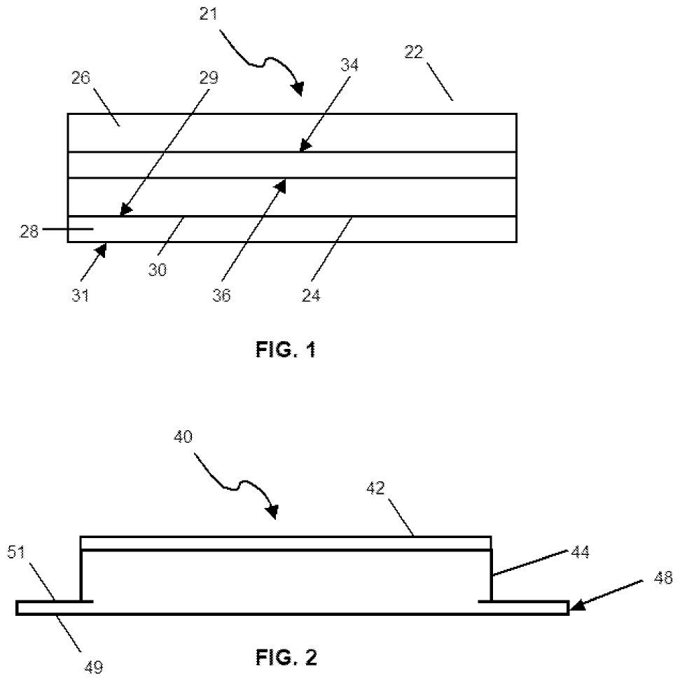

FIG. 1. FIG. 1 shows a reservoir-style dermal patch 10 that comprises a backing 10(i) and a hydrophilic, porous membrane 10(i). The backing 10(i) and hydrophilic, porous membrane 10(i) are attached to one another so as to define a closed volume which acts as a reservoir 10(ii). A preparation comprising a formulation (alternatively called a composition, a pharmaceutical formulation, or a preparation) is disposed in the reservoir 10(ii). First side 10(iii) of the hydrophilic, porous membrane 10(i) is in contact with the formulation. A second side 10(iv) of the hydrophilic, porous membrane 10(i) faces away from backing 10(i) and is coated with a skin adhesive 10(iv). The skin adhesive 10(iv) is preferably formulated to adhere the dermal patch to the user's skin for a period of no less than about twenty four hours while avoiding appreciable skin irritation to the user's skin.

Skin adhesives. The term “skin adhesive” refers to a type of adhesive capable of mediating adhesion of a substrate to human skin of a patient, consumer, or human subject. For example, the substrate can be a dermal patch, a reservoir-style dermal patch, or a monolithic-style dermal patch. Preferably, the skin adhesive can maintain adhesion for at least 24 hours, for at least 48 hours, for at least one week, and so on. Preferably, the skin adhesive can maintain adhesion when the adhesive is exposed to any moisture, such as sweat, released by the skin of the wearer, and can maintain adhesion when the dermal patch is subjected to folding, or warping, or bending, during normal movements of the wearer's arm, torso, thigh, or other site of residence of the dermal patch on the wearer's body.

Preferred skin adhesives 34 include amine-compatible, silicone, pressure sensitive adhesives. In certain examples, an amine-compatible silicone skin adhesive 34 is provided which comprises a trimethylsiloxy end-capped reaction product of a silanol end-blocked polydimethylsiloxane and a silicate resin. The skin adhesive is preferably provided as an organic solvent solution comprising from about 50 percent to about 70 percent by weight of solid adhesive in an organic solvent like heptane or ethyl acetate and having a viscosity at 20 degrees C. of from about 400 mPa-s to about 1300 mPa-s, preferably from about 450 mPa-s to about 1250 mPa-s, and more preferably from about 500 mPa-s to about 1200 mPa-s. A dermal patch may also be called a “device” or a “medical device.”

A first surface 10(iii) of a release liner 10(v) is releasably adhered to skin adhesive 34, and a second surface 10(iv) of release liner 10(v) faces away from skin adhesive 34. Suitable release liners include occlusive polymeric films, such as polyester, polypropylene, coated with a release coating that is releasably adherable to silicone, polyisobutylene, and silicone adhesives. Suitable release coatings on first surface 10(iii) of release liner 10(v) include fluoropolymers and silicone polymers. Commercially-available, coated release liners that are suitable for use as release liner 28 include Scotchpak 1022, 9741, 9744, 9748, and 9755 supplied by 3M of Minneapolis, Minn., and FRA 314 and 315 supplied by Fox River Co. To use the reservoir dermal patch, release liner 10(v) is peeled away from skin adhesive 34, thereby exposing skin adhesive 34, and the dermal patch 10 is applied so that the skin adhesive 34 contacts the user's skin.

More about skin adhesives. Suitable examples of such amine-compatible silicone adhesives include the BIO-PSA 7-4301 and 7-4302 skin adhesives supplied by Dow Corning. BIO-PSA 7-4301 is a high tack, amine-compatible silicone adhesive in heptane available with a solids content of 60 percent and 70 percent and corresponding viscosities at 20 degrees C. of 450 mPa-s and 1600 mPa-s. BIO-PSA 7-4302 is a high tack, amine-compatible silicone adhesive in ethyl acetate with a solids content of 60 percent by weight and a viscosity of 1200 mPa-s at 20 degrees C. The skin adhesive 30 is coated to a thickness per unit area on the membrane 10(i) that is preferably from about 10 to about 20 grams/meter squared more preferably from about 12-18 grams/meter squared, and still more preferably from about 14-16 grams/meter squared.

Hydrophilic, porous membrane 10(i) preferably has a mean flow pore size of no more than about 1 micron, preferably not more than about 0.8 microns, still more preferably no more than about 0.4 microns, and even more preferably no more than about 0.2 microns. At the same time, porous membrane 10(i) preferably has a mean flow pore size of no less than about 0.02 microns, more preferably no less than about 0.04 microns, still more preferably no less than about 0.06 microns, and even more preferably no less than about 0.08 microns. The mean flow pore size may be determined in accordance with the method set forth at page 17, line 22 to page 18, line 4 of published PCT Application WO2010072233.

In the same or other examples, hydrophilic porous membrane 10(i) preferably has a porosity of at least about 60 percent, more preferably at least about 65 percent, and still more preferably at least about 70 percent. At the same time, hydrophilic porous membrane 10(i) preferably has a porosity of no more than about 90 percent, more preferably no more than about 85 percent, and still more preferably no more than about 80 percent. Porosity values may be calculated as described at page 7, lines 24 to 27 of WO2010072233.

In the same or other examples, hydrophilic porous membrane 10(i) preferably has a thickness of no more than about 50 microns, preferably no more than about 40 microns, and even more preferably no more than about 35 microns. At the same time, hydrophilic porous membrane 10(i) preferably has a thickness of no less than about 10 microns, more preferably no less than about 20 microns, and still more preferably no less than about 25 microns. Membrane thicknesses may be determined as described at page 18, lines 19-21 of WO2010072233.

In the same or other examples, hydrophilic porous membrane 10(i) preferably has an air permeability as determined by the Gurley Test Method (according to ISO 5636-5) that is preferably at least about 10 sec/50 mL, more preferably at least about 20 sec/50 mL, and still more preferably at least about 25 sec/50 mL. At the same time, hydrophilic porous membrane 10(i) preferably has an air permeability of no more than about 50 sec/50 mL, more preferably no more than about 40 sec/50 mL, and still more preferably no more than about 35 sec/50 mL.

In the same or other examples, hydrophilic porous membrane 10(i) preferably has a tensile strength in the machine direction as determined by ASTM D882-12 that is preferably at least about 10 MPa, more preferably at least about 15 MPa, and still more preferably at least about 20 MPa. In the same or other examples, the hydrophilic porous membrane 10(i) preferably has a percent elongation in the machine direction as determined by ASTM D882-12 that is preferably at least about 10 percent, more preferably at least about 15 percent, and still more preferably at least about 20 percent.

Hydrophilic porous membrane 10(i) preferably comprises at least one polymeric material. In one example, hydrophilic porous membrane 10(i) comprises a polyolefin polymer and a hydrophilic component that comprises a hydrophilic polymer and optionally, a surfactant. As used herein, the term “hydrophilic” when used to describe a porous membrane refers to a membrane that at 20 degrees C. provides a water flux for demineralized water through the membrane of at least 0.5 liters/(meters squared hbar).

FIG. 2. FIG. 2 discloses an example of monolithic-style dermal patch. Monolithic dermal patch 18 includes a backing 18(i) of the type described previously with respect to backing 18(i) of reservoir dermal patch. A matrix 18(ii) of skin adhesive mixed with a formulation containing one or more pharmaceutical agents is coated on one side of backing 18(i). The matrix 18(ii) is preferably formulated to adhere the dermal patch to the user's skin for a period of no less than about 24 hours while avoiding appreciable skin irritation to the user's skin. A release liner 18(iii) is releasable adhered to matrix 18(ii) on a surface of matrix 18(ii) opposite the surface adhered to backing 18(i). The outer side 18(iii) of release liner 18(iii) faces away from matrix 18(ii) and a portion of innerside 18(iii) of release liner 18(iii) is adhered to matrix 18(ii). To use the monolithic dermal patch, release liner 18(iii) is peeled away and the exposed surface of adhesive matrix 18(ii) is applied to the skin.

The skin adhesive comprising matrix 18(ii) preferably comprises at least one of an acrylate pressure sensitive adhesive, a polyisobutylene pressure sensitive adhesive, and an amine-compatible silicone pressure sensitive adhesive. Suitable acrylate adhesives include DuroTak 87-2074. Suitable polyisobutylene adhesives include those having a viscosity-average molecular weight ranging from about 30,000 Daltons to about 70,000 Daltons, preferably from about 35,000 Daltons to about 65,000 Daltons, and more preferably from about 40,000 Daltons to about 60,000 Daltons. Suitable polyisobutylene adhesives also have a viscosity at 20 degrees C. ranging from about 1000 mPa-s to about 3000 mPa-s. Suitable polyisobutylene adhesives include DuroTak 87-6908.

Adhesion/viscosity modifiers. In certain preferred examples, matrix 18(ii) preferably comprises a polyisobutylene adhesive having a viscosity-average molecular weight as described above and an adhesion/viscosity modifier. The adhesion/viscosity modifier is preferably a mineral oil or silicone fluid present in an amount ranging from about one (1) to about ten (10) percent by weight of matrix 18(ii), more preferably from about two (2) to about six (6) percent by weight of matrix 18(ii), and still more preferably from about three (3) to about four (4) percent by weight of the matrix 18(ii). Mineral oils that are suitable for use as the adhesion/viscosity modifier have a molecular weight ranging from 100 to about 1000 Daltons, more preferably from about 200 to about 600 Daltons, even more preferably from about 350 Daltons to about 450 Daltons, and still more preferably about 400 Daltons. Silicone fluids that are suitable for use as the adhesion/viscosity modifier preferably comprise —OH end-capped polydimethylsiloxanes having a kinematic viscosity at 20 degrees C. ranging from about 100 cSt to about 1000 cSt. Commercially available silicone fluids that may be used as the adhesion/viscosity modifier include the Dow Corning Q7-9120 fluids, which are available in kinematic viscosities (at 20 degrees C.) of 20, 100, 350, 1000, and 12,500 cSt. In preferred examples of silicone adhesion/viscosity modifier, the Q7-9120 100 cSt or 1000 cSt for mixtures thereof) are used.

Polyisobutylene (PIB) adhesives. Preferred polyisobutylene adhesives are not supplied with mineral oil. In certain preferred examples, the polyisobutylene component of matrix 18(ii) is a Vistanex® LM polyisobutylene adhesive. In other preferred examples, the polyisobutylene component of matrix 18(ii) is an Oppanol B13 polyisobutylene adhesive supplied by BASF. BASF has offices in San Diego, CA, Iselin, NJ, and Germany. Vistanex® polyisobutylene is available from ExxonMobil Chemical, Houston, TX.

In yet another example, the adhesive component of matrix 18(ii) may comprise a blend of acrylic adhesive and polyisobutylene adhesive, and preferably, a blend of an acrylic adhesive and a polyisobutylene adhesive having the viscosity-average molecular weight described above (from about 30,000 Daltons to about 70,000 Daltons, preferably from about 35,000 Daltons to about 65,000 Daltons, and more preferably from about 40,000 Daltons to about 60,000 Daltons). When acrylic adhesives are combined with such polyisobutylene adhesives, the amount of acrylic adhesive by weight of the total amount of adhesive in matrix 18(ii) is preferably from about one (1) to about 50 percent. In one example, the adhesive component of matrix 18(ii) comprises 80 percent Oppanol B13 by weight of the total amount of adhesive in matrix 18(ii) and twenty (20) percent Durotak 87-2074 by weight of the total amount of adhesive in matrix 18(ii).

In examples in which matrix 18(ii) comprises an amine-compatible silicone adhesive, the amine-compatible silicone adhesive is preferably of the type described previously with respect to skin adhesive 34 of reservoir dermal patch.

Penetration enhancers. Monolithic dermal patch may also include penetration enhancers, including but not limited to oleic acid, isopropyl palmitate (IPP), DMSO, 1,2 propylene glycol, and isopropyl myristate (IPM). The amount of penetration enhancer preferably ranges from zero to about ten (10) percent by weight of the matrix 18(ii).

Porous membranes, and polymers of said porous membrane. The content of the polyolefin polymer is preferably less than or equal to 98 percent by weight based on the total dry weight of the membrane 10(i), and the content of the hydrophilic component(s) is preferably at least 2 weight percent based on the total dry weight of the membrane. In certain preferred examples, the membrane is formed by combining the polyolefin polymer with the hydrophilic components(s) and optional additives with a solvent to form a blend in the form of a gel, a solution, or a homogeneous mixture, followed by extruding the blend. Suitable polyolefins (such as polyethylene), hydrophilic components, and additives are described in WO2010072233.

One example of a commercially available hydrophilic, porous membrane that is suitable for use as hydrophilic, porous membrane 10(i) is supplied by Lydall Performance Materials B.V. under the name Evopor® 5E02A. Evopor® 5E02A is a porous hydrophilic membrane comprising a polyethylene support and a poly (ethyl vinyl) alcohol hydrophilic component.

Gelling agents. Formulation (composition; preparation) also may comprise a gelling agent which makes the preparation thixotropic. Suitable gelling agents include: sodium carboxymethyl cellulose, hydroxypropyl cellulose, hydroxyethyl cellulose, polyacrylic acid, methyl cellulose, xantham gum, etc. In certain examples, cellulose gelling agents such as hydroxyethyl cellulose are preferred. The gelling agent increases the viscosity of and provides structural integrity to formulation, which improves the ease of placing and retaining formulation in reservoir 10(ii) before the reservoir 10(ii) is closed by heat sealing the hydrophilic, porous membrane 10(ii) to the occlusive backing 10(i). Gelling agents are preferably pharmacologically inactive.

General chemical reagents, as well as cannabinoids, are available (Sigma Aldrich, St. Louis, Mo.; Thermo Fisher Scientific, Waltham, MA, Fisher Chemicals, Fair Lawn, NJ; Cerilliant, Round Rock, TX.; Promochem, Molsheim, France, Cayman Chemical Co., Ann Arbor, Mich.). Purification can be followed by spiking an extract with a labeled cannabinoid. Useful labels include 33P, 35S, 14C, 3H, stable isotopes, fluorescent dyes, or fluorettes (see, e.g., Rozinov and Nolan (1998) Chem. Biol. 5:713-728). Instruments such as high pressure liquid chromatography machines are available from Becton Dickinson, San Jose, CA. Hydrophilic membranes are available, e.g., from Sigma Millipore. Hydrophobic membranes are available, e.g., from Steriltech, Kent, WA.

### DETAILED DESCRIPTIONS OF PREFERRED EMBODIMENTS

Polar organic liquids. Formulation (composition, preparation) of the present disclosure may include a polar organic liquid comprising a molecule having one or more carboxylic acid groups. In the same or other examples, the polar organic liquid comprises a molecule having one or more hydroxyl groups. Suitable polar organic liquids comprising one or more hydroxyl groups include those comprising between 2 and 30 carbon atoms per molecule, including without limitation, ethanol. Suitable polar organic liquids comprising one or more carboxylic acid groups include fatty acids, including without limitation oleic acid. Liquid carriers comprising ethanol and/or oleic acid are preferred, and liquid carriers comprising oleic acid are especially preferred. Suitable liquid carriers also include mixtures of polar organic liquids and water. Examples of such mixtures include mixtures of ethanol and water. In ethanol/water mixtures, the maximum concentration of water is preferably about ten (10) percent by weight of the total amount of ethanol and water.

Antioxidants. Formulation that is enclosed by reservoir of a reservoir-style dermal patch, or a formulation that is included with adhesive of monolithic-style dermal patch can include an antioxidant. Antioxidants may protect cannabinoids, psilocybin, and psilocin, and prevent them from oxidative damage. Antioxidants may include, vitamin C palmitate, vitamin C (also known as, ascorbic acid), alpha-tocopherol (vitamin E), dithiothreitol, dithioerythritol, 2-mercaptoethanol, sulfite and oxygen scavengers.

Other oxygen scavengers are those including an organometallic ligand, where the metal is one of or more of aluminum, antimony, boron, cadmium, calcium, chromium, cobalt, copper, germanium, iron, lead, magnesium, manganese, nickel, palladium, rhodium, scandium, silicon, silver tin, titanium, vanadium, zinc, and combinations thereof.

Methods for measuring oxidation of psilocybin or of psilocin. Methods are disclosed by Hasler, Bourquin, Vollenweider (2002) J. Pharmaceutical and Biochemical Analysis. 30:331-339 and by Martin, Pfeiffer, Kohler (2014) Forensic Sci. Int. 237:1-6. Prophetic efficacy of antioxidative amounts of an antioxidant (such as, ascorbyl palmitate, ascorbic acid, vitamin E, or combinations thereof), can be based on an assumption that zero percent oxidative destruction occurs during storage for under one minute, and that X percent oxidative destruction occurs during storage for seven days, and wherein the desired concentration of antioxidant in the formulation is capable of reducing oxidative destruction to about ninety five (95%) percent of X percent, to about 90% X percent, to about 85% of X percent, to about 80% of X percent, to about 75% of X percent, to about 70% of X percent, to about 65% of X percent, to about 60% of X percent, to about 55% of X percent, to about 50% of X percent, to about 45% of X percent, to about 40% of X percent, to about 35% of X percent, to about 30% of X percent, to about 25% of X percent, to about 20% of X percent, to about 15% of X percent, to about 10% of X percent, or to about 5% of X percent, or to less than 5% of X percent.

To provide a definition of the above parameters, a reduction that is, “to less than 1% of X percent” means that oxidation was almost totally prevented. To provide a further definition, a reduction that is, “to zero % of X percent” means that oxidation was totally prevented.

Liquid carriers and penetration enhancers of the present disclosure. Formulation can comprise a liquid carrier in amounts ranging from about 50 percent to about 99 percent by weight of the formulation. Formulation also comprises from about one (1) percent to about ten (10) percent, and preferably about three (3) percent of a gelling agent (preferably hydroxypropyl cellulose) by weight of the formulation. Formulation also includes from zero to about ten (10) percent by weight of at least one penetration enhancer. In certain examples, the liquid carrier comprises at least one polar liquid. Suitable penetration enhancers include 1,2 propylene glycol, dimethyl sulfoxide (DMSO), oleic acid, and isopropyl palmitate (IPP).

Backings and polymeric films. Suitable backing materials for backing 10(i) include occlusive polymeric films such as polyethylene, polyethylene terephthalate (PET) and combinations thereof. Although the dermal patch of the present disclosure may include an overlay patch, in preferred examples, one is not provided. In general, an overlay patch is not necessary if the hydrophilic, porous membrane 10(i) is already coated with skin adhesive 34. If the membrane 10(i) is not coated with an adhesive (e.g., in order to maximize the flux of pharmaceutical agents into the skin), an overlay patch should be placed over the reservoir 10(ii) in order to ensure intimate contact of the hydrophilic, porous membrane 10(i) with skin. In certain examples, the skin contact area (“active transdermal flux area”) of the membrane 10(i) of a dermal patch is preferably at least about 10 square centimeters, more preferably at least about 20 cm² and still more preferably at least about 30 cm². At the same time, the skin contact area of the dermal patch is preferably no more than about 30 cm², preferably no more than about 25 cm², and still more preferably no more than about 22 cm². At a given flux rate, the skin contact area may be selected to achieve the desired daily dose of pharmaceutical agent (or the dose over whatever time period is of therapeutic interest).

Skin contact area of dermal patch or of reservoir patch. Skin contact area of the dermal patch is preferably at least about 10 square centimeters, more preferably at least about 15 square centimeters, and still more preferably at least about 18 square centimeters. At the same time, the skin contact area of the patch is preferably no more than about 30 square centimeters, preferably no more than about 25 square centimeters, and still more preferably no more than about 22 square centimeters. At a given flux rate, the skin contact area may be selected to achieve the desired daily dose of one or more pharmaceutical agents, such as psilocybin, psilocin, and cannabidiol (CBD) or, alternatively, the dose over whatever time period is of therapeutic interest).

Disorders potentially treatable with formulations comprising psilocybin, with formulations comprising psylocin, or with formulations comprising both psilocybin and psilocin. The following identifies disorders potentially treatable with psilocybin alone, or in combination with one or more of cannabidiol (CBD), other cannabinoids, with or without counseling. The following also identifies disorders potentially treatable with psilocin alone, or in combination with one or more of cannabidiol (CBD), other cannabinoids, with or without counseling. Moreover, the following identifies disorders potentially treatable with the combination of psilocybin and psilocin alone, or with the combination of psilocybin and psilocin with one or more of cannabidiol (CBD), other cannabinoids, with or without counseling.

Disorders include, depression, anxiety/panic disorder, attention deficit hyperactivity disorder (ADHD) or attention deficit disorder (ADD), bipolar disorder, schizophrenia, obsessive compulsive disorder (OCD), autism/Asperger syndrome, antisocial behavior disorder, borderline personality disorder, substance abuse disorder, Tourette's, Parkinson's, epilepsy, migraine, cluster headache, multiple sclerosis (MS), and chronic pain. Symptoms of ADHD and ADD include impairments in attention, impulse control, and hyperactivity. Symptoms of autism/Asperger's include deficits in social communication and interaction, and restricted repetitive behavior (Hutten, Kuypers (2019) Frontiers in Psychiatry. Vol. 10, Article 672). Psilocybin is also being considered for treating reactive anxiety associated with advanced-stage cancer (Grob, et al, supra).

Rating scales for assessing efficacy of psilocybin alone, or in combination with one or more other pharmaceutical agents, with or without counseling. Scales include the Hamilton Anxiety Rating Scale. This scale is used, for example, for measuring clinical status of patients with, “neurotic anxiety states.” The Beck Anxiety Inventory is a 21-item, self-report questionnaire for assessing frequency of anxiety symptoms over a one-week period. This test assesses cognitive symptoms and somatic symptoms (Shear, Vanderbilt, Endicott (2001) Reliability and validity of a structured interview guide for the Hamilton Anxiety Rating Scale. Depression and Anxiety. 13:166-178; Beck, Epstein (1988) J. Consult Clin. Psychol. 56:893-897; Hamilton (1969) Br. J. Psychiatry. 32:50-55). The Beck Depression Inventory consists of questions for measuring the intensity, severity, and depth of depression (Grob et al, supra; Beck, Ward, Mock (1961) 4:561-571). The Profile of Mood States (POMS) is an instrument for assessing feelings and moods of patients (Grob et al, supra; Cella, Jacobsen, Holland (1987) J. Chronic Dis. 40:939-942). The State-Trait Anxiety Inventory (STAI) Form Y is a self-report instrument for assessing anxiety. It has separate measures for state and trait anxiety, and it evaluates feelings of apprehension, tension, nervousness, and worry (Grob et al, supra, Spielberger, Gorsuch, Vagg (1970) State-Trait Anxiety Inventory, Menlo Park, CA: Mind Garden (1970)).

Pain can be measured with the Visual Analogue Scale (VAS). The VAS is a psychometric response scale designed to measure pain distributed over a continuum of values and is not directly measurable. The VAS is comprised of a 10 cm line that ranges from “no pain” on the left (0 cm) to “worst pain” on the right (10 cm) (Younger J, McCue R, Mackey S (2009) Pain outcomes: a brief review of instruments and techniques, Curr. Pain Headache Rep. 13:39-43).

Additional rating scales and instruments include, Hallucinogen Rating Scale (HRS), 5-Dimension Altered States of Consciousness, Mysticism Scale, and the States of Consciousness Questionnaire (SOCQ) (see, Griffiths, Carducci, Klinedinst (2016) J. Psychopharmacology. 30:1181-1197; Griffiths, Richards, McCann (2006) Psychopharmacology. 187:268-283; Hood, Ghorbani, Watson (2001) J. Sci. Study Relig. 40:691-705; ISCDD (2003) GRID-HAMD-17 Structured Interview Guide, San Diego, CA: Int. Soc. for CNS Drug Development; Cohen, Mount, Strobel (1995) The McGill Quality of Life Questionnaire. Palliat. Med. 9:207-219; Dittrich (1998) The standardized psychometric assessment of altered states of consciousness (ASCs) in humans. Pharmacopsychiatry. 31 (Suppl. 2): 80-84). GRID-HAM-A measures anxiety. GRID-HAMD-17 measures depression (Griffiths, Carducci, Klinedinst (2016) J. Psychopharmacology. 30:1181-1197).

Questionnaires for assessing health-related quality of life (HRQoL) in patients are described (see, Tom Brody (2016) Clinical Trials, 2nd ed., Elsevier Press, New York, pages 249-251, 452-473, 480-482, and 559-561). These questionnaires include the Short Form-36 (SF-36).

Designing the control arm for a clinical trial. Typically, a clinical trial is conducted where one arm consists of subjects receiving the experimental drug, and where a second arm takes the form of subjects receiving a control formulation. The control group can receive a formulation consisting of a placebo, such as an excipient or, alternatively, the control group can receive a formulation consisting of a comparator drug (sometimes called an “active control”).

Without implying any limitation, an experimental group can receive a formulation containing psilocybin, cannabidiol (CBD), and excipient where the control group receives a formulation without any psilocybin, where this formulations consists only of a placebo such as the excipient.

Alternatively, an experimental group can receive a formulation containing psilocybin, cannabidiol (CBD), and excipient where the control group receives a formulation without any psilocybin, where this formulation consists of cannabidiol (CBD), and excipient.

To give another example, an experimental group can receive a formulation containing psilocybin, cannabidiol (CBD), and excipient where the control group receives an “active control” formulation without any psilocybin, where this formulation consists of duloxetine, cannabidiol (CBD), and excipient. In this study design, the duloxetine (antidepressant) is the active comparator drug.

To provide one more example, an experimental group can receive a formulation containing psilocybin plus excipient, where the control group receives a control formulation consisting of duloxetine plus excipient. In this study design, the duloxetine (antidepressant) is the active comparator drug.

Controls taking the form of an “active control,” also called a “comparator drug,” are described on pages 78-79, 155-156, 165-169, 223-224, 329, and 484, of Tom Brody (2016) Clinical Trials, 2nd ed., Elsevier Press, San Diego.

### Formulations and Compositions for Delivery by Transdermal Patches

In embodiments, the present disclosure provides a transdermal patch formulation comprising psilocybin, from 1% to 30% and optionally, Vitamin C Palmitate. The disclosure also provides a device for transdermal delivery of psilocybin, comprising a backing; a mixture of an adhesive and a therapeutically effective amount of psilocybin coating on the backing, wherein the mixture includes at least psilocybin, and optionally at least one of tetrahydrocannabinol (THC), tetrahydrocannabinolic acid (THC-a), cannabidiol (CBD), cannabichromene (CBC), a terpene such as beta-caryophyllene; where the adhesive comprises an amino-compatible silicone adhesive and the mixture has a first surface adhered to the backing and a second, skin-adhering surface, a release liner removably attached to the second, skin-adhering surface and the mixture has a first surface adhered to the backing and a second, skin-adhering surface, a release liner removably attached to the second, skin adhering surface of the mixture.

The present disclosure provides transdermal delivery devices for delivering psilocybin through the epidermis of a wearer of the device. Both reservoir-style, and monolithic-style devices are described. In the reservoir style device, a reservoir comprising a solution of psilocybin and a liquid carrier is provided and is defined between a hydrophilic, porous membrane and a backing. One side of the hydrophilic, porous backing is coated with a skin adhesive that is covered with a release liner. To use the device, the user removes the release liner and contacts the skin adhesive-side of the membrane with the skin to affix the device to the body. Preferred skin adhesives include polyisobutylene (PIB) adhesives and amine-compatible silicone adhesives. The phrase “amine-compatible” refers to the fact that the adhesive is substantially non-reactive with amines. Many drugs are amines that bond with hydrogen donors. Silicone adhesive polymers typically have OH groups that are hydrogen donors. When mixed with drugs that have NH groups (amines) or OH groups (like cannabidiol) that can accept hydrogen, the drug and silicone polymer will bond through hydrogen bonding, which retards the diffusion of drug molecules from the adhesive matrix. To avoid that diffusion retardation, the OH groups of the silicone polymer are capped with methyl groups. Such silicone adhesive will be called “amine compatible” which means they are essentially chemically neutral.

The psilocybin diffuses through the hydrophilic porous membrane and the skin adhesive and into the user's skin.

In a first monolithic-style device, a skin adhesive is mixed with the psilocybin to define a substantially monolithic mixture of adhesive and psilocybin. The skin adhesive is coated on a backing that is preferably occlusive. The skin adhesive is preferably a polyisobutylene (PIB) or amine-compatible silicone adhesive.

In a second monolithic-style device, a skin adhesive is mixed with the psilocybin to define a substantially monolithic mixture of adhesive and psilocybin. The skin adhesive is preferably a polyisobutylene adhesive having a viscosity-average molecular weight ranging from about 30,000 Daltons to about 70,000 Daltons, preferably, from about 35,000 Daltons to about 65,000 Daltons, and more preferably from about 40,000 Daltons to about 60,000 Daltons.

Suitable examples of such amine-compatible silicone adhesives include the BIO-PSA 7-4301 and 7-4302 skin adhesives supplied by Dow Corning. BIO-PSA 7-4301 is a high tack, amine-compatible silicone adhesive in heptane available with a solids content of 60 percent and 70 percent and corresponding viscosities at 20 degrees C. of 450 mPa-s and 1600 mPa-s. BIO-PSA 7-4302 is a high tack, amine-compatible silicone adhesive in ethyl acetate with a solids content of 60 percent by weight and a viscosity of 1200 mPa-s at 20 degrees C. The skin adhesive 30 is coated to a thickness per unit area on the membrane 24 that is preferably from about 10 to about 20 g/m², more preferably from about 12-18 g/m², and still more preferably from about 14-16 g/m².

The psilocybin is present in a therapeutically effective amount in the formulation (composition, preparation). A “therapeutically effective amount” is an amount of psilocybin sufficient to achieve a desired therapeutic effect over a desired time period. The psilocybin may be provided as substantially pure psilocybin, such as in a powder form, or as part of an oil extract.

In certain examples, the formulation (composition, preparation) preferably comprises from about one (1) percent to about fifty (50) percent by weight psilocybin, more-preferably from about five (5) to about 30 percent by weight psilocybin, and more preferably from about 10 to about 20 percent by weight psilocybin. In embodiments, in preparation 27, the percent by weight of psilocybin can be 1-2%, 1-4%, 1-6%, 1-8%, 1-10%, or 2-4%, 2-6%, 2-8%, 2-10%, 2-15%, 2-20%, 2-25%, or it can be 5-10%, 5-15%, 5-20%, 5-25%, 5-30%, or it can be 10-15%, 10-20%, 10-25%, 10-30%, 10-35%, or it can be 15-20%, 15-25%, 15-30%, 15-35%, 15-40%, or it can be 20-25%, 20-30%, 20-35%, 20-40%, 20-45%, and the like.

In exclusionary embodiments, a system, a preparation, a device comprising said preparation, a dermal patch comprising said preparation, comprising said declaration, of the present disclosure can exclude any preparation with psilocybin in one of the above ranges, can exclude any device that contains a preparation with psilocybin in one of the above ranges, and can exclude any dermal patch that contains a preparation with psilocybin in one of the above ranges.

In embodiments, the formulation 10(ii) comprises a liquid carrier in amounts ranging from about 50 percent to about 99 percent by weight of the formulation 10(ii). Formulation 10(ii) also comprises from about one (1) percent to about ten (10) percent, and preferably about three (3) percent of a gelling agent (preferably hydroxypropyl cellulose) by weight of the formulation 10(ii). Formulation 10(ii) also includes from zero to about ten (10) percent by weight of at least one penetration enhancer. In certain examples, the liquid carrier comprises at least one polar liquid. Suitable penetration enhancers include 1,2 propylene glycol, dimethyl sulfoxide (DMSO), oleic acid, and isopropyl palmitate (IPP).

Suitable backing materials for backing 10(i) include occlusive polymeric films such as polyethylene, polyethylene terephthalate (PET) and combinations thereof. Although device 10 may include an overlay patch, in preferred examples, one is not provided. In general, an overlay patch is not necessary if the hydrophilic, porous membrane 10(i) is already coated with skin adhesive 34. If the membrane 10(i) is not coated with an adhesive (e.g., in order to maximize the flux of psilocybin into the skin), an overlay patch should be placed over the reservoir 10(ii) in order to ensure intimate contact of the hydrophilic, porous membrane 10(i) with skin. In certain examples, the skin contact area (“active transdermal flux area”) of the membrane 10(i) of a device 10 is preferably at least about 10 cm², more preferably at least about 20 cm² and still more preferably at least about 30 cm². At the same time, the skin contact area of device 10 is preferably no more than about 30 cm², preferably no more than about 25 cm² and still more preferably no more than about 22 cm². At a given flux rate, the skin contact area may be selected to achieve the desired daily dose of psilocybin (or the dose over whatever time period is of therapeutic interest).

EXCLUSIONARY EMBODIMENTS. In exclusionary embodiments, a system, a medical device, a dermal patch, and related methods of the present disclosure can exclude any system, medical device, dermal patch, and related methods that comprises a matrix as disclosed above, an adhesive as disclosed above, a backing as disclosed above, a coating as disclosed above, a polymeric film as disclosed above, a penetration enhancer as disclosed above, or a formulation comprising psilocybin at one of the concentration ranges as disclosed above, and can also exclude any device that contains a preparation with psilocybin in one of the above ranges, and can exclude any dermal patch that contains a preparation with psilocybin in one of the above ranges.

Measuring pain in human subjects. Cannabidiol acts to reduce assonant inflammation (rubor, tumor, calor) as well as chronic pain (dolor).

In studies on human subjects, pain can be rated using the numerical pain rating scale (NPRS), using the Pain Intensity Visual Analog Scale, or using the Pain Relief Verbal Response Scale, and the Global Medication Performance Scale (see, Farrar, Young, Werth (2001) Pain 94:149-158; Farrar, Polomano (2010) Anesthesiology. 112:1464-1472). The Memorial Pain Assessment Scale and the WOMAC pain scale are additional pain scales used with human subjects (see, pages 71 and 232 of Tom Brody (2016) Clinical Trials, 2nd ed., Elsevier Press, San Diego).

Preferred matrices, adhesion/viscosity modifiers, silicone fluids, and adhesive of the present disclosure. In certain preferred examples, matrix 18(ii) preferably comprises a polyisobutylene adhesive having a viscosity-average molecular weight as described above and an adhesion/viscosity modifier. The adhesion/viscosity modifier is preferably a mineral oil or silicone fluid present in an amount ranging from about one (1) to about ten (10) percent by weight of matrix 18(ii), more preferably from about two (2) to about six (6) percent by weight of matrix 18(ii), and still more preferably from about three (3) to about four (4) percent by weight of the matrix 18(ii). Mineral oils that are suitable for use as the adhesion/viscosity modifier have a molecular weight ranging from 100 to about 1000 Daltons, more preferably from about 200 to about 600 Daltons, even more preferably from about 350 Daltons to about 450 Daltons, and still more preferably about 400 Daltons. Silicone fluids that are suitable for use as the adhesion/viscosity modifier preferably comprise —OH end-capped polydimethylsiloxanes having a kinematic viscosity at 20 degrees C. ranging from about 100 cSt to about 1000 cSt. Commercially available silicone fluids that may be used as the adhesion/viscosity modifier include the Dow Corning Q7-9120 fluids, which are available in kinematic viscosities (at 20 degrees C.) of 20, 100, 350, 1000, and 12,500 cSt. In preferred examples of silicone adhesion/viscosity modifier, the Q7-9120 100 cSt or 1000 cSt for mixtures thereof) are used.

Preferred polyisobutylene adhesives are not supplied with mineral oil. In certain preferred examples, the polyisobutylene component of matrix 18(ii) is a Vistanex LM polyisobutylene adhesive. In other preferred examples, the polyisobutylene component of matrix 44 is an Oppanol B13 polyisobutylene adhesive supplied by BASF.

In yet another example, the adhesive component of matrix 18(ii) may comprise a blend of acrylic adhesive and polyisobutylene adhesive, and preferably, a blend of an acrylic adhesive and a polyisobutylene adhesive having the viscosity-average molecular weight described above (from about 30,000 Daltons to about 70,000 Daltons, preferably from about 35,000 Daltons to about 65,000 Daltons, and more preferably from about 40,000 Daltons to about 60,000 Daltons). When acrylic adhesives are combined with such polyisobutylene adhesives, the amount of acrylic adhesive by weight of the total amount of adhesive in matrix 18(ii) is preferably from about one (1) to about 50 percent. In one example, the adhesive component of matrix 18(ii) comprises 80 percent Oppanol B13 by weight of the total amount of adhesive in matrix 18(ii) and twenty (20) percent Durotak 87-2074 by weight of the total amount of adhesive in matrix 18(ii).

In examples in which matrix 18(ii) comprises an amine-compatible silicone adhesive, the amine-compatible silicone adhesive is preferably of the type described previously with respect to skin adhesive 34 of reservoir transdermal device 10.

The amount of psilocybin in the matrix 18(ii) preferably ranges from about one (1) to about 30 percent by weight of the matrix 18(ii), more preferably from about two (2) percent to about 25 percent by weight of the matrix 18(ii), and still more preferably from about five (5) percent to about twenty (20) percent by weight of the matrix 18(ii). The amount of cannabis compounds other than psilocybin is preferably less than about one (1) percent. In those cases where pure psilocybin is used in matrix 18(ii), the amount of pure psilocybin by weight of matrix 18(ii) is preferably from about two (2) percent to about twenty (20) percent, more preferably from about four (4) percent to about fifteen (15) percent, and still more preferably from about five (5) percent to about ten (10) percent.

Monolithic device 18 may also include penetration enhancers, including but not limited to oleic acid, isopropyl palmitate (IPP), DMSO, 1,2 propylene glycol, and isopropyl myristate (IPM). The amount of penetration enhancer preferably ranges from zero to about ten (10) percent by weight of the matrix 18(ii).

In certain examples, the skin contact area of device 10 is preferably at least about 10 cm², more preferably at least about 15 cm², and still more preferably at least about 18 cm². At the same time, the skin contact area of device 10 is preferably no more than about 30 cm², preferably no more than about 25 cm² and still more preferably no more than about 22 cm². At a given flux rate, the skin contact area may be selected to achieve the desired daily dose of psilocybin (or the dose over whatever time period is of therapeutic interest).

### STRUCTURES. Inclusionary Embodiments and Corresponding Exclusionary Embodiments.

The present disclosure can include, or alternatively exclude, an adhesive patch device that has more than one reservoir. The patch device of the present disclosure can have only one reservoir, only two reservoirs, only three reservoirs, only four reservoirs. The present disclosure can exclude microneedles, and can exclude a patch device that has microneedles. The present disclosure can exclude any adhesive patch device, that comprises a bilaminate layer that comprises a trilaminate layer that comprises a tetralaminate layer. Also, the present disclosure can exclude a bilaminate layer, exclude a trilaminate layer, and exclude a tetralaminate layer.

### POLYMERS. Inclusionary Embodiments and Corresponding Exclusionary Embodiments.

System, dermal patch, composition, formulation, and related methods of the present disclosure can include or, alternatively exclude, any system, dermal patch, composition, or formulation that comprises an adhesive polymer, or a device comprising an adhesive polymer, where the adhesive polymer has over 5 free hydroxyl groups per 100 atoms of the adhesive polymer. In embodiments, the adhesive polymer has over 10 free hydroxyl groups per 100 atoms of the adhesive polymer, or it has over 20 free hydroxyl groups per 100 atoms of the adhesive polymer, and so on.

In embodiments, what can be excluded is a monolith-type device where a backing is not in direct contact with a matrix of skin adhesive; where matrix of skin adhesive is not in direct contact with a releasable liner; where matrix does not comprise CBD; or all of the above.

CHEMICALS. Inclusionary embodiments and corresponding exclusionary embodiments. System, dermal patch, composition, formulation, and related methods of the present disclosure can include or, alternatively exclude, any system, dermal patch, patch, or formulation that comprises one or more of sodium phosphate, potassium phosphate, guar gum, gum arabic, locust bean gum, xanthan gum, carrageenan, carob gum, ghatti gum, pectin, tragacanth gum, acacia gum, mannitol, sorbitol, lactose, modified lactose, maltitol, mannitol, magnesium stearate, hydroxypropyl-methylcellulose film, non-crystallizing sugar, or non-crystallizing sugar alcohol.

In embodiments, the present disclosure can exclude a reservoir-type device where backing does not directly contact reservoir; or where reservoir does not directly contact a hydrophilic porous membrane; or where hydrophilic porous membrane does not directly contact a release liner; or where reservoir does not contain all of: (1) a liquid carrier, (2) a gelling agent, and (3) CBD. Also, what can be excluded is a reservoir-type device that does not comprise all of the above.

In embodiments, what can be excluded is an adhesive polymer, or a device comprising an adhesive polymer, where the adhesive polymer reacts with amines. Also what can be excluded, is an adhesive polymer, or a device comprising an adhesive polymer, where the adhesive polymer has any free hydroxyl groups, where the adhesive polymer has over 1 free hydroxyl groups per 100 atoms of the adhesive polymer, where the adhesive polymer has over 5 free hydroxyl groups per 100 atoms of the adhesive polymer, where the adhesive polymer has over 10 free hydroxyl groups per 100 atoms of the adhesive polymer, where the adhesive polymer has over 20 free hydroxyl groups per 100 atoms of the adhesive polymer, and so on. For these exclusionary embodiments the skilled artisan understands that any polymer consists of a large number of atoms, for example, about five thousand atoms.

In embodiments, what can be excluded is a monolith-type device where a backing is not in direct contact with a matrix of skin adhesive; where matrix of skin adhesive is not in direct contact with a releasable liner; where matrix docs not comprise CBD; or all of the above.

What can also be excluded is a preparation, or a device comprising a preparation, where the preparation has over 1% gelling agent, over 2%, over 3%, over 4%, over 5%, over 6%, over 7%, over 8%, over 9%, over 10%, over 12%, over 14%, or over 16%, of gelling agent. Also, what can be excluded is a preparation, or a device comprising a preparation, where the preparation has under 1% gelling agent, under 2%, under 3%, under 4%, under 5%, under 6%, under 7%, under 8%, under 9%, under 10%, under 12%, under 14%, or under 16%, of gelling agent.

### Penetration Enhancers.

In inclusionary embodiments and corresponding exclusionary embodiments, system, device, dermal patch, reservoir-style dermal patch, monolithic-style dermal patch, formulation, composition, reagent, or related method can include (or exclude) any system, device, dermal patch, formulation, composition, reagent, that comprises one or more terpenes (e.g., d-limonene, 1,8-cineole, menthone, menthol, nerolidol, alpha-pinene, beta-carene, see, which is incorporated herein by reference in its entirety), 1-dodecyclazacycloheptane-2-one) (Azone®), oleic acid in propylene glycol, dodecyl-N,N-dimethylamino acetate (DDAA), dodecyl N,N-dimethylamino isopropionate (DDAIP), dimethylsulfoxide (DMSO), dimethylacetamide (DMA), ethanol, butanol, propanol, isopropanol, N-methyl pyrrolidone, lauryl alcohol, fatty acids (e.g., valeric, heptanoic, pelagonic, caproic, capric, lauric, myristic, stearic, caprylic, isovaleric), fatty acid esters, amides, cyclic amides such as Azone, surfactants (e.g., laurate, lauryl sulfate, cetyltrimethyl ammonium bromide, cetylpyridinium, tetradecyl-trimethylammonium), bile salts (cholate, taurocholic, deoxycholic), non-ionic detergents (e.g., Poloxamer®, Brij®, Span®, Tween®), urea, dimethylacetamide, dimethylformamide, a pyrrolidone compound, oxazolidinone, and the like.

Essential oils. In embodiments, a system, dermal patch, reservoir-style patch, monolithic-style patch, formulation, or pharmaceutical composition of the present disclosure can comprise one or more essential oils, such as peppermint oil, orange oil, lemon oil, and so on.

In exclusionary embodiments, a system, dermal patch, reservoir-style dermal patch, monolithic-style dermal patch, formulation, or pharmaceutical composition of the present disclosure can exclude any system, dermal patch, reservoir-style dermal patch, monolithic-style dermal patch, formulation, or pharmaceutical composition that comprises one or more essential oils, such as peppermint oil, orange oil, lemon oil, and so on.

CANNABINOIDS. Inclusionary and exclusionary embodiments. System, device, dermal patch, formulation, composition, and related methods of the present disclosure can include or, alternatively exclude, any system, device, dermal patch, formulation, or composition, that comprises one or more of tetrahydrocannabinolic acid (THCA), cannabidiolic acid (CBDA), and cannabigerolic acid (CBGA), or their decarboxylated their counterparts, tetrahydrocannabinol (THC), cannabidiol (CBD), and cannabigerol (CBG). What can be excluded is delta-9-tetrahydrocannabinol, delta-8-tetrahydrocannabinol, delta-9-tetrahydrocannabinol propyl analogue (THCV), cannabidiol (CBD), cannabidiol propyl analogues (CBDV), cannabinol (CBN), cannabichromene, cannabichromene propyl analogs, and cannabigerol.

More inclusionary and exclusionary embodiments. System, device, dermal patch, formulation, composition, and related methods of the present disclosure can include or, alternatively exclude, any system, device, dermal patch, formulation, or composition, that comprises one or more of cannabigerol; cannabichromene; cannabitriol; cannabidiol; cannabicyclolol; cannabielsoin; cannabinodiol; cannabinol; delta8-tetrahydrocannabinol; delta9-tetrahydrocannabinol; cannabichromanone; cannabicoumaronone; cannabicitnm; 10-oxo-delta6a10a-tetrahydrocannabinol; cannabiglendol; delta7-isotetrahydrocannabinol; CBLVA; CBV; CBEVA-B; CBCVA; delta-9-THCVA; CBDVA; CBGVA; divarinolic acid; quercetin; kaemferol; dihydrokaempferol; dihydroquercetin; cannflavin B; isovitexin; apigenin; naringenin; eriodictyol; luteolin; orientin; cytisosidr; vitexin; cammiprene; 3,4′-dihydroxy-5-methoxy bibenzyl; dihydroresveratrol; 3,4′dihydroxy-5,3′-dimethoxy-5′-isoprenyl; cannabistilbene 1; cannabistilbene 11a; cannabistilbene 11b; cannithrene 1; cannithrene 2; cannabispirone; iso-cannabispirone; cannabispirenon-A; cannabispirenone-B; cannabispiradienone; alpha-cannabispiranol; beta-cannabispiranol; acetyl-cannabispirol; 7-hydroxy-5-methoxyindan-1-spiro-cyclohexane; 5-hydroxy-7-methoxyindan-1-spiro cyclohexane; 5,7-dihydroxyindan-1-cyclohexane; cannabispiradienone; 3,4′-dihydroxy-5-methoxybibenzyl; canniprene; cannabispirone; cannithrene I; cannithrene 2; alpha-cannabispiranol; acetyl-cannabispirol; vomifoliol; dihydrovomifoliol; beta-ionone; dihydroactinidiolide; palustrine; palustridine; plus-cannabisativine; anhydrocannabisativine; dihydroperiphylline; cannabisin-A; cannabisin-B; cannabisin-C; cannabisin-D; grossamide; cannabisin-E; cannabisin-F; cannabisin-G; and so on (see, e.g., Flores-Sanchez and Verpoorte (2008) Secondary Metabolism in Cannabis. Phytochem. Rev. DOI 10.1007).

### EXAMPLES

Formulations of the present disclosure. Formulation the same as previous formulations listed in our U.S. Pat. Nos. 10,799,545 and 10,588,869, but change the active ingredient to a low dose or “microdose” of Psilocin and/or Psilocybine or any derivatives or extracts of the genus Psilocybe. This being for the monolithic patch: ppp ppp ppp

Adhesive Polymer (Silicone, Polyisobutylene, or Acrylate): 60-95%.

Psilocin, Psilocybin, or any derivatives or extracts ofPsilocybe, or any combinations thereof: 0.025-10%, or, more preferably: 0.05-20 mg.

Cannabidiol or other Cannabinoids: 0-20%.

Penetration enhancer: 0-20% (Including, but not limited to, Transcutol, DMSO, Azone, Oleic Acid, Dihydromyricetin, Isopropyl Palmitate, Propylene Glycol, and Isopropyl Myristate)

An antioxidant (including, but not limited to, ascorbyl palmitate): 0.01-1%.

It is important to note that the primary solvent for the dissolution of the actives in this case would be water or methanol rather than ethanol.

In addition, we would like to continue with the “ravioli” reservoir patch, comprised of a solution containing psilocin, psilocybin, or any related compounds derived from Psilocybe along with a liquid carrier with or without a penetration enhancer and antioxidants/antifungals/antibacterials/stabilizers. Further, this would include a a hydrophilic, porous membrane and an occlusive backing, as described in U.S. Pat. No. 10,588,869.

Hydrogels of the present disclosure. Finally, we would like to include hydrogels here. As outlined in U.S. Pat. No. 10,799,545, these hydrogels can include polyvinyl alcohol hydrogel, silicone hydrogel, polyvinyl alcohol/dextran hydrogel, alginate hydrogel, alginate-pyrrole hydrogel, gelatin/chitosan hydrogel, polyacrylic acid hydrogel, photo crosslinked polyacrylic acid, hydrogel, amidated pectin hydrogel, pectin hydrogel, gelatin hydrogel, polyethylene glycol (PEG) hydrogel, carboxymethylcellulose/gelatin hydrogel, chitosan hydrogel, as well as mixtures thereof, or copolymers thereof, and the like. The dosage of Psilocin/Psilocybin or related compounds of Psilocybe in these formulations would remain between 0.05-20 mg, most preferably.

In exclusionary embodiments, a system, monolithic patch, reservoir patch (also called, “ravioli” patch), of the present disclosure can exclude any system, reservoir-style dermal patch, or monolithic-style dermal patch, that comprises one or more of the above hydrogels.

In concentration embodiments, the concentration of hydrogel in the formulation (wt. of hydrogel per gram of formulation) is about 1%, about 2%, about 4%, about 6%, about 8%, about 10%, about 12%, about 14%, about 16%, about 18%, about 20%, about 22%, about 24%, about 26%, about 28%, about 30%, and so on, or is less than about 1%, less than about 2%, less than about 4%, less than about 6%, less than about 8%, less than about 10%, less than about 12%, less than about 14%, less than about 16%, less than about 18%, less than about 20%, less than about 22%, less than about 24%, less than about 26%, less than about 28%, less than about 30%, and so on. In this context, the term “about” can refer to the range of values extending from the next lower value to the next higher value.

In concentration embodiments, the concentration of hydrogel in the formulation (wt. of hydrogel per gram of formulation) is 1%, 2%, 4%, 6%, 8%, 10%, 12%, 14%, 16%, 18%, 20%, 22%, 24%, 26%, 28%, 30%, and so on, or is less than 1%, less than 2%, less than 4%, less than 6%, less than 8%, less than about 10%, less than 12%, less than 14%, less than 16%, less than 18%, less than 20%, less than 22%, less than 24%, less than 26%, less than 28%, less than 30%, and so on.

Derivatives or analogs of psilocin; derivatives or analogs of psilocybin. System, dermal patch, reservoir-style dermal patch, and monolithic-style dermal patch, of the present disclosure, can include a formulation that comprises one or both of derivatives of psilocin and derivatives of psilocybin. The present disclosure provides formulations that comprise a derivative of psilocybin. What is provided is, for example, a derivative that is 1-methyl-psilocin. What is also provided is one or more of compounds 1 to 17 (see, Sard, Morency, Shuster (2005) Bioorganic & Medicinal Chemistry Letters. 15:4555-4559). In exclusionary embodiments, formulations, reservoirs, dermal patches, and systems of the present disclosure can exclude that formulation, can exclude any reservoir, can exclude any dermal patch, and can exclude any system that comprises a derivative (or analog) of psilocybin.

Klein, Laskowski, McCorvy (2021) ACS Pharmacol. Trans. Sci. 2021, 4, 2, 533-542 discloses additional analogs, such as, analogs of psilocybin containing a 4-acetoxy group, such as 4-acetoxy-N,N-dimethyltryptamine. Seventeen different tryptamines, each containing N,N-dialkyl substituents and either a 4-hydroxy or 4-acetoxy group are disclosed. Additional derivatives are, norpsilocin, baeocystin, norbaeocystin, and aeruginascin (Barbara Bauer (April 2019) Dosing With Magic Mushrooms. Psychedelic Science Review.

Mixing procedures of the present disclosure. Step 1. In a 5 liter mixing vessel add ethanol. Step 2. While stirring on a hotplate with a magnetic stirrer (500 rpm) add ascorbyl palmitate. Step 3. When ascorbyl palmitate is dissolved add cannabidiol (CBD). Step 4. Repeat steps 2 and 3 with any and all other active ingredients and excipients. Step 5. Add ethyl acetate and heptane to the mixing vessel. Step 6. Filter solvent solution through quantitative filter paper. Step 7. Add adhesive mixture to 10 liter mixing vessel and place under ultra high-dispersion blade mixer at 2,500 rpm. Step 8. Add solvent solution to adhesive mixture and mix for 10 minutes. Step 9. Increase mixing speed to 3,500 rpm and mix for an additional 20 minutes. Step 10. Place mixture in gyroscopic mixture and mix for 4 minutes, then remove. Step 11. Let mixture sit overnight (preferably in pressure pot), covered, to release any trapped air bubbles.

LAMINATING PROCEDURE. Step 1. Set laminating machine to use flurosilicone coated release liner and foam backing. Set knife blade to 0.007 inches above the top of release liner. Set oven to 85 degrees C. Set web speed to 5 feet per minute, and coating width to 9 inches. Step 2. Using pressure pot at 5 atmospheres, force mixture through coating head and onto web. Once the web has passed through the oven, laminate to backing using lamination head and re-roll on uptake roll. Step 3. Once lamination step is complete, let roll sit for at least 24 hours.

CUTTING PROCEDURE. Step 1. Feed laminated roll through kiss-cut die-cutting machine. Step 2. Cut laminated roll to patches of the size, 50 cm².

INGREDIENTS AND CONCENTRATIONS. Cannabidiol (CBD) concentrations between 1 and 25%, preferably between 5-20%, more preferably between 10-20%, more preferably between 15-20%. Capsaicin concentrations between 0.01-2%, more preferably between 0.1-1%, more preferably 0.1-0.5%, more preferably 0.1-0.25%. Menthol concentrations between 0.1-15%, more preferably between 1-10%, more preferably 2.5-5%. Lidocaine concentrations between 0.1-5%, more preferably between 2.5-5%, more preferably 4% in an OTC product and 5% in a prescription-only product. Ascorbyl palmitate at 0.1% w/w acts as an antioxidant in this formulation, and has been present in our previous patents in the same manner. Diclofenac concentrations between 0.1-2%, more preferably 0.75-1.5%, more preferably between 1-1.25%. Camphor concentrations between 0.1-15%, more preferably between 1-10%, more preferably between 2.5-5%. Methyl Salicylate concentrations between 0.1-15%, more preferably between 2.5-15%, more preferably between 5-10%.

EXCIPIENTS. The only excipient would be the 6% Transcutol (diethylene glycol monoethyl ether), which is used here as a permeation enhancer. The solvent used for the active ingredient (and Transcutol) dissolution is Ethanol, for the adhesive, Heptane, and as a cosolvent, Ethyl Acetate. These solvents are removed via evaporation in an oven, and residual solvent concentrations for each should be no higher than 50 micrograms per gram.

PERMEATION ENHANCERS. The only permeation enhancer is Transcutol at 6% w/w. The adhesive mixture makes up 79.9% of the final patch weight (excluding the backing and the release liner). Of that 79.9%, 85% is DuPont Liveo BIO-PSA AC7-4301 and 15% is DuPont Liveo BIO-PSA AC7-4201.

HYDROGELS. Technically no matrix is included, since it is a drug-in-adhesive patch. Systems, compositions, devices, dermal patches, and related methods, of the present disclosure may include formulations that comprise a hydrogel.

Hydrogels are 3-dimensional, cross-linked networks of water-soluble polymers. The porous structure of hydrogels can be altered by changing the density of cross-linking. The degree of cross-linking can alter the rate of loading a drug, and it can alter the rate of drug release. The present disclosure can encompass a hydrogel that consists of one of the following polymers or alternatively, that comprises one or more of the following polymers (e.g., as a block polymer). The polymers include, poly(ethylene oxide) (PEO), poly(propylene oxide) (PPO), poly(lactide-co-glycolic acid) (PLGA), poly(N-isopropylacrylamide) (PNIPAM), poly(propylene fumarate) (PPF), poly(caprolactone) (PCL), poly(urethane) (PU), and poly(organophosphazene) (POP). An example of a block polymer is PEO—PPO-PEO. In exclusionary embodiments, the present disclosure can exclude a hydrogel that includes PEO, PPO, PLGA, PNIPAM, PPF, PCL, PU or POP. The present disclosure also encompasses hydrogels that contain a cyclodextrin, where the cyclodextrin is cross-linked to hydrogel (see, Hoare et al (2008) Hydrogels in drug delivery: Progress and challenges. Polymer. 49:1993-2007). Hydrogels of the present disclosure can be ethylene vinylacetate, alginic acid, gums, polyvinylalcohol hydrogel; silicone hydrogel; polyvinylalcohol/dextran hydrogel; alginate hydrogel; alginate-pyrrole hydrogel; gelatin/chitosan hydrogel; polyacrylic acid hydrogel; photo crosslinked polyacrylic acid hydrogel; amidated pectin hydrogel; pectin hydrogel; gelatin hydrogel; polyethylene glycol (PEG) hydrogel; carboxymethylcellulose/gelatin hydrogel; chitosan hydrogel, as well as mixtures thereof, or copolymers thereof, and the like. Hydrogel with crosslinks are available (Lee et al (2003) Eur. J. Pharm. Biopharm. 56:407-412.

Printing Active Ingredients and Excipients on Dried Hydrogels. Dried hydrogel can take the form of a “xerogel” or of a film. Xerogel can be made by freeze drying a hydrogel. Film can be made by evaporative drying or casting from organic solutions. Spotting device can be used to apply microdrops in predetermined locations of dried hydrogel or on a film (see, e.g., U.S. Pat. No. 6,642,054 of Schermer). Where dried hydrogel or film takes the form of a layer, microdrops can be applied to one side only or to both sides. Where more than one type of drug is to be applied and where at least two of the drugs are incompatible with each other, or where a drug an excipient are to be applied, and where these are incompatible with each other, these can be applied at different locations on the dried hydrogel or on the film. (Drop size of microdrops can be, e.g., 0.05 nanoliters (nL)-10,000 nL, 0.5 nL-200 nL, 10 nL-100 nL, and so on. Drug, active ingredient, and/or excipient is not incorporated into the dried hydrogel, but is instead printed on its surface or surfaces. Printing on dried hydrogel avoids problems arising from incompatibility of drug, active ingredient, and/or excipient with the hydrogel itself. See, US 2008/0095848 of Stabenau, which is incorporated by reference in its entirety.

CYCLODEXTRINS. Cyclodextrins are cyclic oligosaccharides of (alpha-1,4)-linked alpha-D-glucopyranose units, with a lipophilic central cavity and a hydrophilic outer surface. As a result of their molecular structure and shape, they can act as molecular containers by trapping drugs or other molecules in their internal cavity. No covalent bonds are formed or broken during drug cyclodextrin complex formation, and in aqueous solution, the complexes readily dissociate and free drug molecules remain in equilibrium with the molecules bound within the cyclodextrin cavity (see, Tiwari et al (2010) Cyclodextrins in delivery systems: Applications. J. Pharm. Bioallied Sci. 2:72-79). Derivatives of cyclodextrins that are hydroxypropyl (HP), methyl (M) and sulfobutylether (SBE) substituents are useful as pharmaceutical excipients.

Cyclodextrins for use, for example, in cannabinoid/cyclodextrin complex, include beta-cyclodextrins such as hydroxypropyl-beta-cyclodextrin, sulfobutylether-beta-cyclodextrin, maltoxyl-beta-cyclodextrin, and methylated cyclodextrins. Encompassed are alpha-cyclodextrins (6 glucopyranose units), beta-cyclodextrins (7 glucopyranose units), and gamma-cyclodextrins (8 glucopyranose units). Methylated cyclodextrins can improve aqueous solubility, dissolution rate, and bioavailability of cannabinoids.

The present disclosure provides a dermal patch comprising a dextrin where the dextrin is not complexed with a pharmaceutical agent, and a dermal patch comprising a dextrin where the dextrin is, in fact, complexed with a pharmaceutical agent.

In exclusionary embodiments, the present disclosure can exclude a formulation that comprises a cyclodextrin, or that comprises an alpha-cyclodextrin, or that comprises a beta-cyclodextrin, or that comprises a gamma-cyclodextrin. What can also be excluded is a device that comprises a cyclodextrin, such as an adhesive dermal patch comprising a dextrin.

APERTURES AND PORES. The present disclosure can encompass films, sheets, layers, membranes, and the like, including those with a plurality of apertures or pores. In some aspects, the apertures or pores have an average diameter of 20 nm, 40 nm, 50 nm, 100 nm, 200 nm, 300 nm, 400 nm, 500 nm, 600 nm, 800 nm, 0.001 mm, 0.002, 0.005 mm, 0.010 mm, 0.015 mm, 0.020 mm, 0.025 mm, 0.030 mm, 0.040 mm, 0.050 mm, 0.075 mm, 0.10 mm, 0.20 mm, 0.30 mm, 0.40 mm. 0.50 mm, and the like. Also, the pores can have a diameter range where the range is bracketed by any two of these values. In other aspects, the apertures or pores have a diameter in the range of 20-40 nm, 40-60 nm, 60-80 nm, 50-100 nm, 100-200 nm, 200-400 nm, 400-600 nm, 600-800 nm, 800-1,000 nm, 0.001-0.002 mm, 0.001-0.005 mm, 0.005-0.010 mm, 0.010-0.020 mm, 0.020-0.040 mm, 0.025-0.050 mm, 0.050-0.075 mm, 0.075-0.10 mm, 0.10-0.20 mm, 0.20 mm-0.40 mm, 0.25-0.50 mm, 0.50-0.75 mm, 0.50-1.00 mm, 0.1-0.2 mm, and so on. In exclusionary embodiments, the present disclosure can exclude films, sheets, layers, and the like, that have apertures or pores having any of the above average values, or that are describable by any of the above ranges.

Porous membranes can take the form of hydrophilic porous membranes and hydrophobic porous membranes, without implying any limitation. Hydrophobic membranes, such as hydrophobic polyethylene (PE) membranes, can be made more hydrophilic by alcohol or surfactants (see, WO2010/072233 of Calis). Pores in membranes of the present disclosure can have an average diameter of about 5 micrometers, about 10, about 15, about 20, about 25, about 30, about 40, about 50, about 60, about 70, about 80, about 90, about 100, about 110, about 120, about 130, about 140, about 150, about 160, about 170, about 180, about 190, or about 200 micrometers, and the like. Also, pores in the membranes can have an average diameter somewhere in the range 5-20 micrometers, 20-40 micrometers, 40-60 micrometers, 60-80 micrometers, 80-100 micrometers, 100-120 micrometers, 120-140 micrometers, 140-160 micrometers, 160-180 micrometers, 180-200 micrometers, and so on. In exclusionary embodiments, the present disclosure can exclude any membrane that is characterized by one of the above “about” values or that is characterizable by one of the above ranges.

For any given film, sheet, or layer, and the like, the area of a plurality of apertures or the area of a plurality of pores can occupy about 1%, about 2%, about 4%, about 6%, about 8%, about 10%, about 15%, about 20%, about 25%, about 30%, about 35%, about 40%, about 45%, about 50%, about 55%, about 60%, about 65%, about 70%, about 75%, about 80%, about 85%, about 90%, and the like of the surface area. In exclusionary embodiments, the present disclosure can exclude any film, sheet, or layer, where the area does not occupy one or more of the given percentage values, or where the area does not occupy a range between any two of the above given percentage values. The above parameters also can apply to a film, sheet, or layer, with perforations, where the value of the area for the perforation is measured flush with a surface of the film, sheet, or layer.

MACHINES. The machines used include: An ultra high shear dispersion blade mixer; A hotplate stirrer and magnetic stir bar; A gyroscopic mixer; A custom manufactured lamination machine that uses the solvent casting method to lay down the mixed adhesive formulation to a knife-over-roll coating head. The machine also includes a 5′ oven which, for this formulation, maintains a temperature of 85 degrees C. The web moves at 5′ per minute, resulting in an oven time of exactly 1 minute. The web is then laminated to an occlusive backing and rerolled; A custom manufactured die-cutting machine which kiss-cuts the patches to a variable shape and size including, but not limited to circles, ovals, squares, rectangles, rounded-edge squares, rounded edge-rectangles, hexagons, rounded-edge hexagons, and other polygons with or without rounded edges of sizes 10 cm², 15 cm², 20 cm², 25 cm², 30 cm², 35 cm², 40 cm², 45 cm², 50 cm², 55 cm², 60 cm², 70 cm², 80 cm², 90 cm², 94 cm², and 100 cm².

### Manufacturing Processes and Equipment.

Sealing Two Strips Together at the Edges, and Coordinating Transverse Sealing to Create Pouches and Filling of the Pouches. What is provided is a method to feed two strips into a machine with rollers to move the strips at the same speed, and to cause the two strips to move downwards, where the first face of the first strip is caused to contact the first lace of the second strip. The first face is caused to contact the first face of the second strip, in preparation for heating the edges of the two strips, thus sealing the two strips together, and in preparation for transverse heating, with heating at intervals of distance and time, thus creating a plurality of pockets in the sandwich of the two strips. When the two strips are moved downwards, the first strip and second strip are situated to form a thicker sandwich that moves downwards. Heaters resembling wheels or rollers, clamp down on the edges of the 2-strip sandwich, causing the 2-strip sandwich to form a long, closed tube. While the 2-strip sandwich moves downwards, what simultaneously occurs is simultaneous heating/sealing of a pair of transverse clamps. The transverse clamps create separate pouches in the long 2-strip sandwich. When the heated bars clamp down, what is created is a top seal of a previously-filled pouch, and the bottom seal of a pouch that has yet to be filled. Simultaneously occurring with heating/scaling at the edges by the heated wheels, and simultaneously occurring with heating by the transverse bars, is filling of each pouch as it is created, where filling is by a long tube that reaches down into the long sandwich to fill each pouch as it is created. See, U.S. Pat. No. 6,871,477 of Tucker, which is incorporated by reference in its entirety. The first strip can comprise an adhesive layer and permeable membrane, the second strip can be an impermeable backing, and the gel can comprise a cannabinoid in gel form.

Unrolling Three Different Layers From Rolls, Stripping Off Release Liners From Two of the Layers, Aligning the Three Layers Together to Form a Complex, and Rolling the Complex on to a Roll. The present disclosure provides machinery that can unroll a plurality of rolls, optionally with stripping off a release-layer from one or more of the rolls, and taking up the stripped-off release-layer on an empty rotating drum or roll. For example, three different rolls can contain three different laminates, the first laminate comprising: (1) Protective backing; (2) Combined zone of transport enhancement and zone of containment; and (3) Release layer. The second laminate can comprise; (1) Adhesive layer; (2) Zone of transport control; and (3) Release liner. And the third laminate can comprise: (1) Support film; (2) Adhesive; and (3) Removable liner that is not removed during the above-mentioned method. Machinery can include three rolls on three rotating mechanisms of first, second, and third laminate, respectively. Machinery can include take-up rolls lor taking up release liners. Machinery can include a pair of rollers situated on opposite sides of moving sandwich of first laminate and second laminate for use in bringing the two laminates together. Also, machinery can include a pair of rollers situated on opposite sides of the nascent 3-part sandwich, where the 3-part sandwich takes the form of the combined (in contact with each other) first and second laminate and the entering third laminate. The entering third laminate is simultaneously unrolled from its roll and then combined with the complex of first and second laminate. The final product is then moved, by way of pairs of rollers situated on opposite sides of the moving final product, and also moved by way of individual rollers, e.g., rollers called over roller, under roll, and over idler roller. The above-disclosed machinery can also include a device for sealing laminates together, a corona discharge for enhancing the sealing of the laminates together, a device for depositing a drug or adhesive or other composition on one or more of the laminates as the laminate is unrolled from its roll, and cutting devices for separating the sandwich of three laminates into patches. See, U.S. Pat. No. 5,370,924, which is incorporated herein by reference in its entirety.

Layered devices may be assembled and then sealed by vacuum forming or by heat sealing without vacuum. In exclusionary embodiment, the present disclosure can exclude machinery, methods, and patches, made using one or more of vacuum forming, heat sealing, corona discharge, one or more crimp rolls, or cooperating nip.

Providing a Platen with Bar-Like Regions Separated by Channels, and Using the Platen to Stamp a Laminate, and to Provide Pressure on Regions That Need to be Collapsed, While Refraining From Providing Pressure on Regions That Contain Drug and Matrix. The present disclosure provides machinery, such as a platen with bar-like regions separated by channels, and where the bar-like regions are optionally heated. The platen can be used to selectively compress parts of a laminate, where the laminate (the “workpiece”) comprises an upper layer that is a cellular region and a lower layer that is a skin adhesive. The platen selectively compresses the distal sides (the right edge and left edge), resulting in collapse of the distal sides of the laminate. Optionally, only the part of the laminate destined to be collapsed is provided with the adhesive. The cellular region can be reticulated or it can be non-reticulated. The cellular region can be made of foamed thermoplastic resin. Cell size can be about 0.05, about 0.1, about 0.2, about 0.4, about 0.6, about 0.80, about 1.0, about 1.2, about 1.4, about 1.6, about 1.8, about 2.0, about 2.5, about 3.0, or about 4.0 millimeters. Collapsed regions are such that drugs cannot easily pass through collapsed regions. In embodiments, non-collapsed central area (the area that resided under the channel during platen-manufacturing process) can contain a distally-situated layer of drug-releasing matrix (which contains drug) in contact with a proximally-situated layer of a medium through which drug can diffuse. The layer of medium through which drug can diffuse can be, e.g., gel, cream, or ointment. “Distal” means away from the skin when patch is attached to skin, and “proximal” means on the side of patch that is closest to skin, when patch is attached to skin. The compressed lateral pans of patch may be called “straps.” See, U.S. Pat. No. 5,505,958 of Bello, which is incorporated herein by reference in its entirety.

In exclusionary embodiments, the present disclosure can exclude patch devices with non-compressed cellular region, patch devices with compressed cellular region, layered structures with a distally-situated drug matrix and a proximally-situated gel, cream, ointment, or other medium through which drug can diffuse on its way to skin. Also, the present disclosure can exclude any composition, laminate, layered structure, and patch that was made via heating of a layered structure or via heating of a laminate.

Placing Drug Between Two Webs, Sealing Two Webs Together, Crimping the Sealed Webs to Form Pockets and Cutting the Sealed Web. The present disclosure provides machinery and methods for using, as starting material, two different webs, each on a roller, where each web comprises one or more of a film, adhesive layers, impermeable layers, porous layers, and the like. The finished product takes the form of the two webs that are sealed together, and where an active ingredient, such as a composition comprising one or both cannabinoid and terpene, is contained therein. In the method, a first supply roll provides one web and a second supply roll provides a second web. Machinery at various “stations” modify one of the webs or modify both of the webs, as the webs move along a conveyor belt. One station, which is optional, is a corona discharge station. The corona discharge modifies the surface chemistry of one or both of the webs, prior to marriage of the two webs together by operation of two crimp rolls. The corona discharge modifies the surface chemistry to improve adhesive properties of the first web and/or of the second web. Corona discharge is preferred where dissimilar materials (one material of first web, and other material of second web) are to be adhesively joined. Dissimilar materials can be, e.g., polyester polymer and ethylene acrylic acid polymer.

Another station is deposit station, which deposits active substance on one of the webs, as the web moves towards the crimp roll. Deposit station can include a reservoir that contains drug and tube leading from reservoir to location on web surface where drug is to be deposited. The deposit station preferably occurs after the corona station. Also, the deposit station and corona station preferably act on the same web, though optionally deposit station can operate on first web and corona station can operate on second web. The two webs are securely fastened together in a station taking the form of a first crimp roll and a second crimp roll. These rolls resemble gears, in that first crimp roll has projections and second crimp roll has depressions, which act meashingly in the manner of a “tongue-and-groove” to compress the two webs together and, at the same time, to stamp the joined webs into a pocket-like shape. The regions of the first crimp roll and second crimp roll that mesh together are called a “cooperating nip.”

Finally, after the webs pass through the corona station, drug deposit station, and crimp rolls, the joined webs are cut by rotary die cutter, to create flexible packages or flexible patches suitable for marketing. Motors can drive rollers. Also, motors can drive crimp rolls. See, U.S. Pat. No. 4,782,647 of Williams which is incorporated herein by reference.

Separating Cut Patches From a Strip of Non-Cut Patches, and Transferring Cut Patches to a Carrier. This describes only one step in the procedure for making adhesive patches, e.g., monolithic devices and reservoir devices. The procedure involves a cutter, transfer devices resembling wedges, and rollers. The rollers function to move a first web and a second web, in the manner of a conveyor belt. The first web takes the form of an auxiliary layer film on top, and then just under it, a drug-containing adhesive layer that sits on top of a carrier film. The first web, which has these three layers, is then later on supplemented by a process layer, where the result is a web consisting of four layers (process layer on top, then auxiliary layer film, then drug-containing adhesive layer, and on the bottom, carrier film). An earlier-occurring cutting process has cut the auxiliary layer film and the drug-containing adhesive layer into blocks. The first web is moved in one direction, e.g., to the left, and then with the help of the transfer devices resembling wedges, the squares are separated from the carrier film (the carrier film is then moved away to the right) and also separated from the combination of auxiliary layer film and process layer (which is moved upwards), where the squares end up residing on a carrier film. At this point the blocks are separated from each other, and any scrap that had been created with the cutting process is then discarded. This refers to the situation where cutting creates discrete blocks and creates scraps in between the blocks. The supporting film supports the blocks and moves away to the left. See, U.S. Pat. No. 6,059,913 of Asmussen, which is incorporated herein in its entirety.

Cutting Laminate to Create Fully Cut-Out Region and, Within it, a Partially Cut (Scored) Region. Machinery, methods, and workpiece of the present disclosure comprises sheet of laminate, where shapes of the sheet (rectangles, ovals, circles) are cut fully through the laminate, and where the edges of the cut-out laminate is called, “periphery” (outer cut). Where the cut-out laminate is circular, the periphery is the same as the circumferential region. In addition to being cut at the periphery, the sheet is simultaneously cut during the cutting operation in a region within the periphery (inner cut). The inner cut has a smaller diameter than the outer cut. Also, the inner cut is to a shorter depth than the outer cut. In the case of a 3-layer laminate (release layer; pressure-sensitive adhesive, backing), the outer cut slices through all three layers, but the inner cut slices only partially through the top layer (the release layer). This partial cutting is more properly called, “scoring” rather than “cutting.” The goal of this 2-distance cutting method is to score the release layer to facilitate easy removal of the liner by the user, and at the same time, to avoid leaking of adhesive from the patch during storage of the patch. Machinery for the 2-distance cutting method can take the form of a roller covered with cutting stampers (similar to cookie-cutters). Each cookie cutter stamps all the way through the laminate.

Within each cookie cutter resides a second (smaller diameter) cookie cutter which is sized so that it only cuts partially through the top layer of the laminate (thus only scoring the top layer). In an alternative machinery, a first roller bears an array of only the larger diameter (and longer cutting distance) cookie cutters, while the second roller bears an array of the smaller diameter (and scoring distance) cookie cutters. In operation, the two rollers operate simultaneously, and the cookie cutters on the first roller are aligned exactly with the cookie cutters on the second roller and, in operation the cutting (cutting through all layers) occurs simultaneously with scoring, for each patch. See, U.S. Pat. No. 5,656,285 of Sablotsky, which is incorporated by reference in its entirety, in addition to the one cutting roller (or to the two cutting rollers), the machinery can have a pressure roller and a support roller, for use in driving the sheet of laminate. In exclusionary embodiments, the present disclosure can exclude an adhesive dermal patch that has a scored region, such as a scored release layer.

Efficient Separation of Punched Patches From Scrap Web. During manufacture of adhesive patches, patches are stamped out from, or cut from, a sheet consisting of various layers. These layers may include backing, matrix containing a drug, skin adhesive, and release layer. During cutting, some of the punched patches that have not yet been separated from the scrap web may cling to the scrap web as the scrap web is pulled away from the sheet. Where this clinging is maintained as the scrap web is pulled away, the result will be undesirable discarding of the clinging punched patches along with the scrap web. This type of undesired clinging can be increased by flow of adhesive out of the edges of the punched patch, followed by flow of the adhesive to contact the scrap web. Efficient separation of punched patches can be accomplished by way of a probe or probes that contact the punched patch and shove the punched patch onto a horizontally moving conveyor belt as the scrap web is drawn upwards for eventual discard. The probe can take the form of a rotating roller where the roller is covered with blocks having the same shape and exactly the same dimensions (or dimensioned to be about 5% smaller, about 10% smaller, about 15% smaller, about 20 smaller, and the like, in area, as compared to the punched patch). The blocks can have a shape, as viewed from “above,” that is square, rectangular, oval, round, etc., and to have a shape corresponding to the punched patch. Thus, as the roller rotates, each block presses down on a corresponding punched patch (as the punched patch continues to move on the conveyor belt) while the scrap web is simultaneously detached and drawn upwards by the rotation of the roller. An alternative to using a roller covered with block probes, is a roller covered with flexible bristles. As the roller rotates, the bristles press springfully down on the punched patches, the bristles remaining bent, causing the punched patches to separate from the scrap web. At the same time, the bristles pressing on the scrap web are greatly bent at first, but as the scrap web is pulled upwards, the bristles spring out to their full (un-bent) length. See, US 2017/0136648 of Grader, which is incorporated herein by reference in its entirety. In an exclusionary embodiment, the present disclosure can exclude manufacturing machinery and methods, comprising a roller with blocks or a roller with bristles, for use in preventing punched patches from adhering to the scrap web.

FRANZ CELL. Flux is preferably measured using human cadaver skin with a Franz Diffusion Cell (see, Kristof (2017) Feasibility of transdermal delivery of Cyclosporine A using plasma discharges. Biointerphases. 12:02B402). Flux can be measured where the temperature of the Franz Diffusion Cell (and solutions inside the Franz Diffusion Cell) are held constant at about 17 degrees C., about 20 degrees C., about 23 degrees C. (“room temperature”), about 26 degrees C., about 30 degrees C., about 33 degrees C., about 37 degrees C. (mammalian body temperature), about 40 degrees C., and so on.

Time Period for Measuring Flux. In embodiments, flux of a chemical, with transfer from a dermal patch to human skin, can be measured where flux is allow to continue uninterrupted over a period of time that is, about ten minutes, about 20 minutes, about 30 minutes, about 40 minutes about 50 minutes, about 60 minutes, about 1 hour, about 1.5 hours, about 2 hours, about 3 hours, about 4 hours, about 5 hours, about 6 hours, about 7 hours, about 8 hours, about 9 hours, about 10 hours, and the like. In a preferred embodiment, cumulative flux over a designated time period is measured. In other embodiments, the period of time is ten minutes, 20 minutes, 30 minutes, 40 minutes 50 minutes, 60 minutes, 1 hour, 1.5 hours, 2 hours, 3 hours, 4 hours, 5 hours, 6 hours 7 hours, 8 hours, 9 hours, about 10 hours, and the like.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention

What is claimed is:

1. A reservoir-type dermal patch that includes:
(i) A backing and a hydrophilic, porous membrane,
(ii) Wherein the backing and hydrophilic, porous membane are attached to one another to define reservoir, wherein the reservoir comprises a pharmaceutical formulation,
(iii) Wherein the hydrophilic, porous membrane comprises a first surface that is on the side of the hydrophilic, porous membrane that contacts the backing,
(iv) Wherein the hydrophilic, porous membrane further comprises a second surface that is on the side of the hydrophilic, porous membrane that does not contact the backing and, wherein the second surface is coated with a skin adhesive,
(v) Wherein the dermal patch further comprises a release liner that contacts the second surface that is coated with the skin adhesive,
(vi) And wherein the pharmaceutical formulation comprises one or both of psilocybin and psilocin.

2. The dermal patch of claim 1, wherein the formulation further comprises one or more antioxidants,
said one or more antioxidants consisting of an antioxidatively effective concentration of an ascorbyl palmitate, or consisting of an antioxidatively effective concentration of ascorbic acid, or consisting of a combination of both an antioxidatively effective concentration of ascorbyl palmitate plus ascorbic acid,
wherein the formulation includes psilocybin, and wherein said one or more antioxidants is capable of reducing the rate of oxidation of psilocybin.

3. The dermal patch of claim 1, wherein the formulation further comprises one or more antioxidants, wherein said one or more antioxidants comprises an antioxidatively effective concentration of an ascorbyl palmitate,
or an antioxidatively effective concentration of ascorbic acid,
or a combination of both an antioxidatively effective concentration of ascorbyl palmitate plus ascorbic acid,
wherein the formulation includes psilocin, and wherein said one or more antioxidants is capable of reducing the rate of oxidation of psilocin.

4. The dermal patch of claim 1, wherein regarding the formulation, the sum of the concentrations of psilocybin and psilocin is one of about 0.02% by weight (unit of wt./wt.), about 0.04% by weight, about 0.06% by weight, about 0.08% by weight, about 1.0% by weight, about 2.0% by weight, about 4% by weight, about 6% by weight, about 8% by weight, about 10% by weight, about 12% by weight, about 14% by weight, or about 16% by weight,
wherein the unit of weight is weight of psilocybin in a gram of formulation were the formulation does not contain psilocin,
or weight of psilocin in a gram of formulation where the formulation does not contain psilocybin,
or weight of the sum of psilocybin plus psilocin where the formulation contains both psilocybin and psilocin.

5. The dermal patch of claim 1, wherein the reservoir contains a hydrogel.

6. The dermal patch of claim 1, wherein the reservoir does not contain any hydrogel.

7. The dermal patch of claim 1, wherein the reservoir contains a penetration enhancer, and wherein the penetration enhancer is at a concentration of about 2% (wt./wt.), about 4%, about 6%, about 8%, about 10%, about 12%, about 14%, about 16%, about 18%, or about 20%.

8. The dermal patch of claim 1, wherein the reservoir does not contain any penetration enhancer, or wherein the reservoir contains under one percent penetration enhancer (wt./wt.).

9. The dermal patch of claim 1, wherein the formulation contains one or more penetration enhances selected from transcutol, dimethylsulfoxide (DMSO), azone, oleic acid, dihydromyricetin, isopalmitate, propylene glycol, and isopropyl myristate.

10. The dermal patch of claim 1, wherein the formulation comprises psilocin and one or more cannabinoids.

11. The dermal patch of claim 1, wherein the formulation comprises psilocybin and one or more cannabinoids.

12. The dermal patch of claim 1, wherein the formulation comprises both psilocin and psilocybin and one or more cannabinoids.

13. A monolithic-type dermal patch that includes:
(i) A backing,
(ii) A matrix comprising a skin adhesive, an adhesion/viscosity modifier, and a pharmaceutical formulation, wherein the pharmaceutical formulation comprises one or more of psilocybin, psilocin, a derivative of psilocybin, a derivative of psilocin, a cannabinoid, and an antioxidant,
(iii) A release liner, wherein the release liner has an inner face and a outer face, and wherein the inner face is substantially in contact with the matrix, wherein the outer face does not contact the matrix, and wherein said release liner can be peelably removed from the surface of said adhesive matrix,

14. The monolithic-type dermal patch of claim 13, wherein the adhesion/viscosity modifier is a mineral oil or a silicone fluid.

15. The monolithic-type dermal patch of claim 14, wherein the mineral oil or the silicone fluid is present in an amount ranging from zero percent to about ten percent of the weight of the matrix.

16. The monolithic-type dermal patch of claim 14, wherein the mineral oil has a molecular weight ranging from 200-400 Daltons, from 200-500 Daltons, from 300-500 Daltons, from 300-600 Daltons, from 400-600 Daltons, from 400-700 Daltons, from 500-700 Daltons, from 500-800 Daltons, from 600-800 Daltons, from 600-900 Daltons, from 700-900 Daltons, from 700-1000 Daltons, or from any combination of said ranges.

17. The monolithic-type dermal patch of claim 14, wherein the silicone fluid comprises hydroxyl group (—OH) end-capped polydimethylsiloxanes having a kinematic viscosity at 20 degrees C. ranging from 100 cSt to about 1000 sCt, or wherein the kinematic viscosity at 20 degrees C. is about 20 cSt, or about 100 cSt, or about 350 cSt, or about 1000 cSt, or about 12,500 cSt.

18. The monolithic-type dermal patch of claim 13, wherein the skin adhesive is a polyisobutylene that is supplied in mineral oil, or wherein the skin adhesive is a polyisobutylene that is not supplied in mineral oil.

19. The monolithic-type dermal patch of claim 13, wherein the skin adhesive comprises a blend of acrylic adhesive and polyisobutylene adhesive.

20. The monolithic-type dermal patch of claim 13, wherein the skin adhesive an amine-compatible silicone adhesive.

21. The monolithic-type dermal patch of claim 13, that comprises a penetration enhancer, and wherein said penetration enhancer comprises one or more of oleic acid, isopropyl palmitate (IPP), DMSO, 1,2 propylene glycol, and isopropyl myristate (IPM).

22. The monolithic-type dermal patch of claim 21, wherein the amount of penetration enhancer preferably ranges from zero to about ten percent by weight of the matrix.
